(19) United States
(12) Patent Application Publication
(10) Pub. No.: US 2020/0281950 A1
(43) Pub. Date: Sep. 10, 2020

(54) LIPOSOMES FOR TREATMENT OF AN AUTOIMMUNE DISEASE

(71) Applicant: MB BIOTECH LTD., Pardes- Hanna (IL)

(72) Inventor: Moshe BARU, Pardes-Hanna (IL)

(73) Assignee: MB BIOTECH LTD., Pardes-Hanna, OT (IL)

(21) Appl. No.: 16/066,125

(22) PCT Filed: Dec. 27, 2016
(86) PCT No.: PCT/IL2016/051383

§ 371 Date: Jun. 26, 2018

(51) Int. Cl. A61K 31/685; A61K 9/127; A61P 1/00

(52) U.S. Cl. CPC A61K 31/685; A61P 1/00; A61K 9/1271

(57) ABSTRACT

Compositions and methods utilizing drug-free liposomes for treatment of autoimmune diseases and in particular inflammatory bowel disease such as ulcerative colitis and Crohn's disease are provided.

### FIELD OF THE INVENTION

The present invention provides compositions and methods utilizing liposomes for treatment of autoimmune diseases. In particular, the liposomes are empty liposomes lacking any additional or encapsulated active agent.

### BACKGROUND OF THE INVENTION

Inflammatory Bowel Disease (IBD) is an idiopathic chronic inflammation of gastrointestinal (GI) tract. Several types of IBD are known: Ulcerative colitis (UC) is limited to the mucosal layer of the colon; Crohn's disease (CD) is inflammation involving the full thickness of any part of the GI tract and indeterminate colitis, which is present in 15% patients with IBD, which is impossible to differentiate.

According to some research the incidence of IBD is 10 cases per 100,000 per year (in the USA). It is estimated that there are about 1.3 million cases of IBD in the US alone. UC is more prevalent in males, and CD in females. The peak of disease onset occurs at ages 20-30 years for CD and 30-40 years for UC. About 7-20% of the IBD patients are pediatric cases.

Symptoms of the disease depend on extent and severity of inflammation. UC symptoms include bloody diarrhea, abdominal cramping, rectal tenesmus, i.e. fecal urgency, systemic symptoms such as fever, decreased stamina and weight loss. One third of the patients experience extra-intestinal manifestations. Crohn's disease symptoms include diarrhea, chronic abdominal pain, weight loss, fever, perianal disease and extra-intestinal manifestations. The symptoms may vary with type and location of disease (structuring, fistulizing).

The treatment of IBD is mostly symptomatic and depends on the severity of the disease. It includes aminosalicylates, oral steroids, corticosteroids, immunomodulatory drugs, such as azathioprine (AZA) and 6-mercaptopurine (6-MP) and biologic therapy such as Infiximab or Adalimumab (monoclonal antibodies against TNF-α), cyclosporine (immunosuppressant drug) and surgery in severe cases (see FIG. 1).

However the current available treatments have many limitations Aminosalicylates are only modestly effectives and have significant side effects. Glucocorticoids cause severe adverse events and do not provide benefits as maintenance therapy. Immune suppressants (6-mercaptopurine, azathioprine) have slow onset of action and serious side effects including 4 times increased risk of lymphoma. Tumor necrosis factor alpha (TNF-α) antagonists predispose patients to serious infection including tuberculosis (TB) and fungal infections, and do not present clinically relevant effect in all treated patients. Between 20% and 40% of CD patients will not respond to induction therapy with TNF-α inhibitors and about 40% of patients will lose response to TNF-α inhibitors over time. Additional drawbacks of biological therapy (such as treatment with monoclonal anti-TNF-α antibodies) is that anti-drug antibodies may develop infusion reactions and injection site reactions may occur and the cost of treatment high.

A liposome is a spherical vesicle having at least one phospholipid bilayer surrounding an aqueous solution core. Liposomes are generally divided into categories based on their composition, number of bilayers and size, ranging from small unilamellar vesicles to large multilamellar vesicles. Liposomes having polyethylene glycol (PEG) on their surface are commonly referred to as PEGylated liposomes. PEGylation significantly reduces opsonization and prolongs the circulation half-life of liposomes. The production of liposomes is relatively simple and reproducible and the cost of manufacture is relatively low. Typically, PEG-liposomes are stable at 2-8° C. degrees for at least two years.

It has been shown that PEGylated liposomes (PEG-liposomes) of less than 200 nm accumulate in inflamed tissues. It was further shown that PEG-liposomes penetrate and concentrate in inflamed colon of rats (Awasthi et al., J Drug Target. 2002; 10(5):419-27). It was also demonstrated that PEG-liposomes attach to white blood cells (WBC) following in-vivo administration (Constantinescu et al., Artif Cells Blood Substit Immobil Biotechnol. 2003 November; 31(4): 395-424).

Kesisoglou et al. (Pharmaceutical Research, 2005, 22 (8) 1320-1330) demonstrated that efficiency of encapsulation of 6-MP into liposomes was dependent on lipid content and composition. While liposomal encapsulation significantly reduced systemic absorption of 5-ASA and increased its tissue levels due to liposomal adherence to intestinal tissue this was not the case for 6-MP; local tissue levels of 6-MP were not improved compared to solution drug.

U.S. Pat. No. 8,252,836 disclose a unique composition of a 5-amino salicylic acid (5ASA) and a phospholipid for treating Inflammatory Bowel Disease (IBD), where the composition can be a mixture, a molecular association complex or a covalent compound of 5ASA and a reactive phospholipid covalently bonded together via a diazo linkage and to methods for administering the compositions to treat symptoms of IBD.

WO 2013/066179 discloses a pharmaceutical composition comprising liposomes composed of non-charged vesicle-forming lipids, optionally including not more than 10 mole percent of negatively charged vesicle-forming lipids and/or not more than 10 mole percent of PEGylated lipids, the liposomes having a selected mean particle diameter in the size range of 40-200 nm and comprising a first corticosteroid in water soluble form, for the site-specific treatment of inflammatory disorders in humans.

There is a clear medical need for an improved, cost-effective treatment for inducing and maintaining remission of IBD that has robust efficacy and minimal side effects.

### SUMMARY OF THE INVENTION

The present invention provides methods for treating an autoimmune disease in a subject in need thereof, said method comprising administering a pharmaceutically acceptable composition comprising a therapeutically effective amount of liposomes, wherein said composition is substantially devoid of a compound being an active agent in a medication prescribed for treatment of the autoimmune disease. According to some embodiments, the treated autoimmune disease is IBD.

According to the present invention, pharmaceutical compositions comprising empty small unilamellar vesicle (SUV) liposomes, i.e. liposomes that are substantially devoid of any additional or encapsulated active agent, are effective in treating and suppressing gastrointestinal autoimmune disease such as inflammatory bowel disease (IBD). It is now disclosed for the first time, that the treatment of IBD with empty PEGylated liposomes, significantly suppresses various pathological manifestations of colitis, including weight loss, intestinal bleeding and diarrhea, resulting in substantial reduction of disease activity.

The present invention is based in part on the experimental finding that the treatment with empty PEG-liposomes reduced the colon damage in mice with induced colitis; as shown herein below, the colons of treated animals were significantly longer than the colons of untreated ones.

Without wishing to be bound by any theory or mechanism of action it is proposed that empty small unilamellar vesicle (SUV) PEGylated liposomes accumulate in inflamed tissues, bind to white blood cells and reduce inflammation. The liposomes are relatively easy to produce, stable, and the cost of their manufacture is relatively low. Clinical experience with PEG-liposome products indicate that no significant side effects are expected. It is encompassed according to the principles of the present invention that liposomes, and in particular empty liposomes, may be used for treatment of IBD either as a single therapy or as part of a regimen in combination with other drugs.

In one aspect the present invention provides a method of treatment of a gastrointestinal autoimmune disease in a subject in need thereof, said method comprises administering a pharmaceutically acceptable composition comprising a therapeutically effective amount of SUV liposomes, wherein said composition is substantially devoid of a compound being an active agent in a medication prescribed for treatment of a gastrointestinal autoimmune disease. According to some embodiments, the treated gastrointestinal autoimmune disease is IBD.

Thus in one embodiment, the present invention provides a method of treatment of IBD in a subject in need thereof said method comprising administering a pharmaceutically acceptable composition comprising a therapeutically effective amount of SUV liposomes, wherein said composition is substantially devoid of a compound being an active agent that is a medication prescribed for treatment of IBD disease. According to some embodiments, the liposomes are PEGylated liposomes. The liposomes or the PEGylated liposomes according to the present invention and pharmaceutical composition comprising said liposomes may comprise an active agent, with the proviso that the liposomes and/or composition are substantially devoid of a compound being an active agent in a medication prescribed for treatment of an autoimmune disease. According to particular embodiments the liposomes and/or pharmaceutical composition are substantially devoid of steroids, corticosteroids, anti-inflammatory agents, any active agent in a prescription drug, compounds prescribed for treatment of an autoimmune diseases or compounds used for treatment of IBD, and specifically compounds prescribed for treatment of gastrointestinal autoimmune diseases, Crohn's disease or ulcerative colitis. According to some embodiments, the liposomes according to the present invention comprise no active agent being a prescription drug. In further embodiments, the liposomes are empty liposome, i.e. drug-free liposomes. The liposomes according to the present invention may further comprise non-active compounds such as vitamins, minerals, buffers, antioxidants, nutraceuticals, antioxidants, or pharmaceutically acceptable excipients.

According to another aspect, the present invention provides a method of treatment of an autoimmune disease in a subject in need thereof, said method comprising administering a pharmaceutically acceptable composition comprising a therapeutically effective amount of SUV liposomes, wherein said composition is substantially devoid of a compound being an active agent in a medication prescribed for treatment of any autoimmune disease. According to some embodiments, the treated autoimmune disease is IBD.

According to certain aspects, the present invention provides a pharmaceutically acceptable composition comprising SUV liposomes, for use in treatment of a gastrointestinal autoimmune disease, wherein said composition is substantially devoid of a compound being an active agent in a medication prescribed for treatment of a gastrointestinal autoimmune disease. According to one embodiment, the composition is substantially devoid of a compound being an active agent in a medication prescribed for treatment of an autoimmune disease.

According to another aspect, the present invention provides a pharmaceutically acceptable composition comprising SUV liposomes, for use in treatment of a gastrointestinal autoimmune disease, wherein said composition is substantially devoid of a compound being an active agent in a medication prescribed for treatment of any autoimmune disease.

### BRIEF DESCRIPTION OF THE FIGURES

FIG. 1 shows the therapeutic pyramid for active ulcerative colitis and Crohn's disease.

FIG. 2 shows the effect of a treatment with MB-102 on the weight loss in mouse colitis model.

FIG. 3 shows the effect of a treatment with MB-102 on the Disease Activity Index in in mouse colitis model.

FIG. 4 show the colon length and colon's content of healthy mice (FIG. 4A) and mice with induces colitis: treated with Saline (control) (FIG. 4B) or treated with MB-102 (FIG. 4C).

FIG. 5 show the effect of a treatment with MB-102 on the colon length in mice with induced colitis.

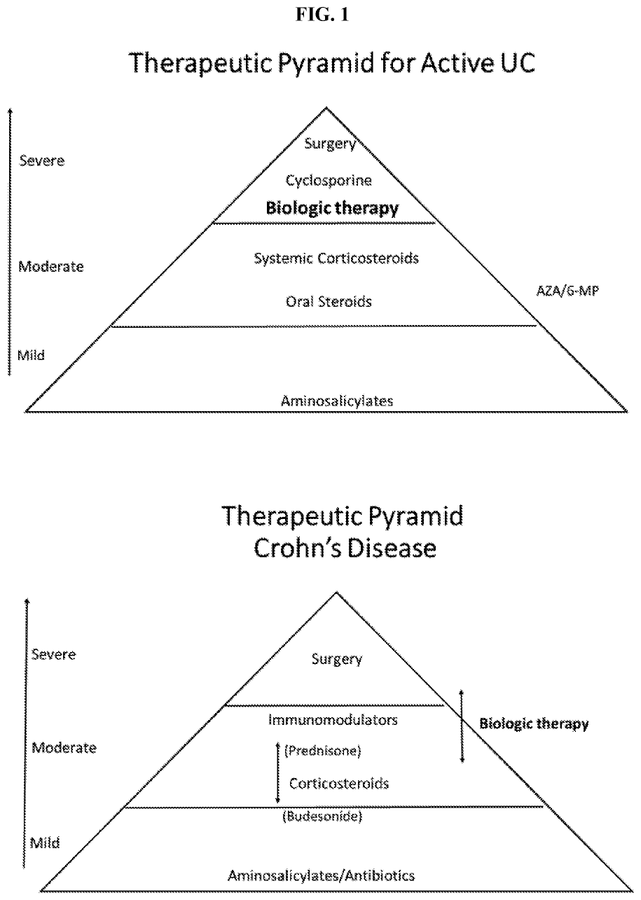

### DETAILED DESCRIPTION OF THE INVENTION

According to one aspect, the present invention provides a method of treatment of an autoimmune disease in a subject in need thereof comprising administering a pharmaceutically acceptable composition comprising a therapeutically effective amount of small unilamellar vesicle (SUV) liposomes, wherein said composition is substantially devoid of a compound being an active agent in a medication prescribed for treatment of an autoimmune disease. According to one embodiment, the present invention provides a method of treatment of a gastrointestinal autoimmune disease in a subject in need thereof comprising administering a pharmaceutically acceptable composition comprising a therapeutically effective amount of SUV liposomes, wherein said composition is substantially devoid of a compound being an active agent in a medication prescribed for treatment of a gastrointestinal autoimmune disease. According to one another, the present invention provides a method of treatment of a gastrointestinal autoimmune disease in a subject in need thereof comprising administering a pharmaceutically acceptable composition comprising a therapeutically effective amount of SUV liposomes, wherein said composition is substantially devoid of a compound being an active agent in a medication prescribed for treatment of an autoimmune disease.

The terms “treating” or “treatment” as used herein refers to taking steps to obtain beneficial or desired results, including clinical results. Beneficial or desired clinical results include, but are not limited to, alleviation or amelioration of one or more symptoms associated with autoimmune disease, delay or slowing of that disease, amelioration, palliation or stabilization of that disease, in particular gastrointestinal autoimmune disease, and other beneficial results. According to some embodiment, treating refers to downregulating the activity of a marker of inflammation and/or expression of at least one gene coding for the marker of inflammation in peripheral blood lymphocytes of the patient. In certain embodiment, treating refers to improving and/or ameliorating the symptoms of a gastrointestinal autoimmune disease. In one embodiment, this term refers to alleviation or amelioration of one or more symptoms associated with IBD, such as bloody diarrhea, abdominal cramping, rectal tenesmus, i.e. fecal urgency, fever, decreased stamina, weight loss diarrhea, chronic abdominal pain, perianal disease and extra-intestinal manifestations. In one embodiment, the term refers to treatment of IBD.

The term “effective amount” as used herein refers to a sufficient amount of the liposomes that, when administered to a subject, will have the intended therapeutic effect.

“Administering” or “administration of” a substance, a compound, an agent or a composition to a subject can be carried out using one of a variety of methods known to those skilled in the art. For example, a compound, an agent or a composition can be administered enterally or parenterally. Enterally refers to administration via the gastrointestinal tract including per os, sublingually or rectally. Parenteral administration includes administration intravenously, intradermally, intramuscularly, intraperitoneally, subcutaneously, ocularly, sublingually, intranasally, by inhalation, intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. According to some embodiments, the administration is oral administration. According to other embodiments, the administration is parenteral administered, e.g. by injection or by infusion. According to further embodiments, the administration is rectal administration. According to further embodiments, the administration is oral administration.

The method according to the present invention encompass administering a pharmaceutical composition comprising SUV liposomes with a proviso that said composition and said liposomes are substantially devoid of a compound being an active agent in a medication prescribed (e.g. require physician approval) for treatment of an autoimmune disease such as gastrointestinal (GI) autoimmune disease.

The term “pharmaceutical composition” and “pharmaceutically acceptable composition” are used herein interchangeably and refer to a composition comprising liposome as disclosed herein formulated together with one or more pharmaceutically acceptable carriers. Such composition may further comprise one or more active agent which is not the active agent in a medication prescribed or used for treatment of an autoimmune disease such as GI autoimmune disease.

The terms “pharmaceutically acceptable carrier” or “pharmaceutically acceptable excipient” as used herein refer to any and all solvents, dispersion media, preservatives, antioxidants, coatings, isotonic and absorption delaying agents, surfactants, buffer and the like, that are compatible with pharmaceutical administration. The use of such media and agents for pharmaceutically active substances is well known in the art. The compositions may contain other active agent s providing supplemental, additional, or enhanced therapeutic functions.

The terms “autoimmune disease” or “autoimmune disorder” are used herein interchangeably and refer to any disease or disorder that occurs when the body's immune system erroneously attacks and destroys healthy body tissue. Examples for such autoimmune disease are digestive autoimmune disease, multiple sclerosis or lupus. In some embodiments, the autoimmune disease is rheumatoid arthritis. In some embodiments the autoimmune disorder is other than rheumatoid arthritis and condition associated therewith. In some embodiments, the autoimmune disease is any autoimmune disorder with a proviso that the term does not include rheumatoid arthritis, osteoarthritis, or osteoarthritis in rheumatoid arthritis. According to some embodiment, the autoimmune disease is a gastrointestinal autoimmune disease or digestive autoimmune disease, such as ulcerative colitis, Crohn's disease, celiac disease irritable bowel syndrome or autoimmune hepatitis. The terms “gastrointestinal autoimmune disease” and “digestive autoimmune disease” are used herein interchangeably and refer to autoimmune disease of a gastrointestinal tract. The term “gastrointestinal tract” includes mouth, oesophagus, stomach, and intestines. The intestines refers to small intestine which includes duodenum, jejunum, ileum and to the large intestine which includes cecum, colon, rectum, and anal canal. In one embodiment, the autoimmune disease is Crohn's disease. In another embodiment, the disease is ulcerative colitis. In a further embodiment, the disease is irritable bowel syndrome.

The terms “substantially devoid”, “essentially devoid”, “devoid”, “does not include” and “does not comprise” may be used interchangeably and refer to composition that does not include, contain or comprise a particular compound, e.g. said composition comprises less than 0.1 mol %, less than 0.01 mol %, or less than 0.001 mol % of the compound.

Liposomes:

The term “liposome” as used herein refers to a spherical vesicle having at least one lipid bilayer. Liposomes for use in this invention may be prepared to include liposome-forming lipids, e.g. phospholipids and lipopolymers, and optionally membrane active sterols, e.g. cholesterol. According to some embodiments the liposomes that may be used in accordance with the invention, do not include in their bilayers a membrane active sterol, such as cholesterol. A membrane active sterol is defined as affecting short- and long-range lipid order within membranes, minimizing volume, and decreasing membrane permeability.

Liposome-forming lipids are mainly glycerophospholipids and sphingomyelins. The glycerophopsholipids include lipids having a glycerol backbone wherein at least one, preferably two, of the hydroxyl groups at the head group is substituted by one or two of an acyl, an alkyl or alkenyl chain, a phosphate group, an acyl chain (to form an acyl or diacyl derivative), a combination of any of the above, and/or derivatives of same, and may contain a chemically reactive group (such as an amine, acid, ester, aldehyde or alcohol) at the headgroup, thereby providing a polar head group. The sphingomyelins have two hydrocarbon chains one is an acyl chain covalently bound to the primary amino group of the long chain amino alcohol sphingosine that contributes the second long hydrocarbon chain. Phosphocholine attached by a phosphoester to the Cl hydroxyl group of the sphingosine contributes the polar head group of the sphingomyelin (which is identical to this group of phosphatidylcholine).

The liposome forming lipids are typically, 14-24 carbon atoms in length, and have varying degrees of saturation, thus resulting in fully, partially or non-hydrogenated liposome-forming lipids. The lipids may be of a natural source, semi-synthetic or fully synthetic lipids, and may be neutral, negatively or positively charged. There are a variety of synthetic liposome forming lipids and naturally-occurring liposome forming lipids, including phospholipids, such as phosphatidylcholine (PC), phosphatidylinositol (PI), phosphatidylglycerol (PG), dimyristoyl phosphatidylglycerol (DMPG), egg yolk phosphatidylcholine (EPC), 1-palmitoyl-2-oleoylphosphatidyl choline (POPC), hydrogenated soy phosphatidylcholine (HSPC), distearoylphosphatidylcholine (DSPC), dimyristoyl phosphatidylcholine (DMPC); phosphatidic acid (PA), phosphatidylserine (PS); 1-palmitoyl-2-oleoylphosphatidyl choline (POPC), and the sphingophospholipids such as sphingomyelins (SM) including N-stearoyl sphingomyelin, or N-palmitoyl sphingomyelin. The above-described lipids and phospholipids can be obtained commercially or prepared according to published methods in the art.

The liposomes of the present invention may further comprise glyceroglycolipids, sphingoglycolipids and sterols (such as cholesterol or plant sterol).

The liposome-forming lipids can be in the form of zwitterionic phospholipids wherein for example the cationic and anionic moieties have identical number of charges (constituting the head group) all remain fully ionized over a broad pH range with no net charge (zeta potential=˜0 mV). Cationic lipids (mono- and polycationic) may also be used to form liposomes either as an individual component or wherein the cationic lipid is included as a minor/major component of the lipid composition. Such cationic lipids typically have a lipophilic moiety, such as a sterol, an acyl or diacyl chain, and where the lipid has an overall net positive charge.

Liposomes may include other phospholipids, such as phosphatidylethanolamines and their hydrophilic polymer derivatives. The lipopolymers may be non-ionic lipopolymers (also referred to as neutral lipopolymers or uncharged lipopolymers) or lipopolymers having a net negative or positive charge.

The liposomes may also include a lipid derivatized with a polymer to form new entities known by the term lipopolymers. The head group may be polar or apolar. The hydrophilic polymer head group may be attached to the lipid region covalently or non-covalently; however, it is preferably attached via the formation of a covalent bond (optionally via a linker). The outermost surface coating of the hydrophilic polymer chains is effective to provide a liposome with a long blood circulation lifetime in vivo. While the lipids derivatized into lipopolymers may be neutral, negatively charged, or positively charged, the most commonly used and commercially available lipids derivatized into lipopolymers are those based on phosphatidyl ethanolamine (PE), usually, distearylphosphatidylethanolamine (DSPE). A specific family of lipopolymers which may be employed by the invention include monomethylated PEG attached to DSPE (with different lengths of PEG chains, the methylated PEG referred to herein by the abbreviation MPEG) in which the PEG polymer is linked to the lipid via a carbamate linkage resulting in a negatively charged lipopolymer. Other lipopolymers are distearoyl, i.e. lipopolymers comprising two stearoyl moieties. Examples of such lipopolymers are distearoylglycerol and polyethyleneglycol oxycarbonyl-3-amino-1,2-propanediol distearoylester. Such lipopolymers may be PEGylated, e.g. neutral methyl polyethyleneglycol distearoylglycerol (MPEG-DSG) and the neutral methyl polyethyleneglycol oxycarbonyl-3-amino-1,2-propanediol distearoylester (MPEG-DS).

As used herein, the content of liposomes refers to the lipid fraction of the liposome including modifications thereof, unless stated otherwise. Therefore, wherein stated that the liposome comprises X mol % of a particular component, it should be understand that the lipid fraction of the liposome comprise X mol % of that component. The terms “mol percent” and “mol %” as used herein are well known in the art and refer to the percentage of a particular component to all reactants, as measured in molar percent.

According to some embodiments, the liposomes of the present invention comprise about 30 mol % to about 100 mol %, about 40 mol % to about 90 mol %, about 50 mol % to about 80 mol %, or about 60 mol % to about 70 mol % of liposome-forming lipids.

According to some embodiments, the liposomes of the present invention comprises phosphatidylcholine, such as egg yolk phosphatidylcholine (EPC), 1-palmitoyl-2-oleoylphosphatidyl choline (POPC), hydrogenated soy phosphatidylcholine (HSPC), distearoylphosphatidylcholine (DSPC) or dimyristoyl phosphatidylcholine (DMPC). In some embodiments, the lipid liposomes comprise about 30 mol % to about 100 mol %, about 40 mol % to about 90 mol %, about 50 mol % to about 80 mol %, or about 60 mol % to about 70 mol % of phosphatidylcholine. According to more particular embodiments, the liposome comprise hydrogenated soy phosphatidylcholine (HSPC), e.g about 30 mol % to about 100 mol %, about 40 mol % to about 90 mol %, about 50 mol % to about 80 mol %, or about 60 mol % to about 70 mol % of HSPC.

According to some embodiments, the liposomes of the present invention may comprise stabilizing polymer molecules. According to some embodiment, the stabilizing polymer molecules are bound to a liposome forming molecule. According to another embodiment, the stabilizing polymer molecules are bound to lipopolymers. According to certain embodiments, the stabilizing polymer molecules are bound to the surface of liposomes. There are numerous polymers, which may be used in liposomes according to the present invention. Polymers typically used as lipid modifiers include, but not limited to: polyalkylether such as polyethylene glycol (PEG), polysialic acid, polylactic acid (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. In one embodiment, the polymer is PEG or modified PEG such as methylated PEG (MPEG) and more specifically monomethylated PEG.

According to any one of the above embodiment, the liposomes of the present invention comprise stabilizing polymer molecules. According to one embodiment, the polymer is selected from polyalkylether, polysialic acid, polylactic acid and polyglycolic acid. According to one embodiment, polyalkylether is polyethylene glycol. Therefore according to some embodiments, the liposomes according to the present invention are PEGylated liposomes, i.e. a liposomes comprising PEG molecules. According to one embodiment, the PEG moiety attached to the liposomes according to the present invention has an average molecular weight of about 350 Da to about 100,000 Da, about 500 Da to about 40,000 Da, about 750 Da to about 20,000 Da, about 1,000 Da to about 12,000 Da, or about 1500 Da to about 10,000 Da, about 2000 Da to about 8000 Da, about 3000 Da to about 6000 Da, about 4000 Da to about 5000 Da or about 2000 Da to about 4,000 Da. According to a further embodiment, the PEG moiety has an average molecular weight of about 1500 to about 3000 Da. According to some embodiment, the stabilizing polymer molecules are bound to a liposome forming molecule and/or to lipopolymers.

According to some embodiment, the liposome comprise about 0 mol %, e.g. about 0.001 mol %, about 0.01 mol % or about 0.1 mol % to about 20 mol %, about 0.5 mol % to about 18 mol %, about 1 mol % to about 16 mol %, about 3 mol % to about 15 mol %, about 5 mol % to about 14 mol %, about 7 mol % to about 13 mol %, about 8 mol % to about 12 mol %, or about 10 mol % of PEG. According to a further embodiment, the liposomes comprise about 2 mol % to about 10 mol %, or about 4 mol % to about 8 mol %, or about 5 mol % to about 7 mol % of MPEG-DSPE.

According to some embodiments, the liposomes comprise phosphatidylethanolamine such as distearylphosphatidylethanolamine (DSPE). According to some embodiments, phosphatidylethanolamine is PEGylated phosphatidylethanolamine, i.e. phosphatidylethanolamine conjugated with PEG molecules. According to one embodiment, the liposomes comprise PEGylated DSPE. According to some embodiments, PEG has an average molecular weight of about 350 Da to about 100,000 Da, about 500 Da to about 40,000 Da, about 750 Da to about 20,000 Da, about 1,000 Da to about 12,000 Da, or about 1500 Da to about 5,000 Da or about 2000 Da to about 4,000 Da. According to more particular embodiments, the PEGylated DSPE is methyl-polyethyleneglycol distearylphosphatidylethanolamine (MPEG-DSPE). According to some embodiments, PEG of the MPEG-DSPE has an average molecular weight of about 350 Da to about 100,000 Da, about 500 Da to about 40,000 Da, about 750 Da to about 20,000 Da, about 1,000 Da to about 12,000 Da, or about 1500 Da to about 5,000 Da or about 2000 Da to about 4,000 Da. According to a further embodiment, the MPEG moiety has an average molecular weight of about 1500 to about 3000 Da. According to a still another embodiment, the PEG moiety has an average molecular weight of 2000 Da. According to some embodiments, the liposome comprise about 0 mol %, e.g. 0.001 mol %, 0.01 mol % or 0.1 mol % to about 20 mol %, about 5 mol % to about 15 mol %, or about 10 mol % of MPEG-DSPE. According to another embodiment, the liposome comprise about 0.5 mol % to about 18 mol %, about 1 mol % to about 16 mol %, about 3 mol % to about 15 mol %, about 5 mol % to about 14 mol %, about 7 mol % to about 13 mol %, about 8 mol % to about 12 mol %, or about 10 mol % of MPEG-DSPE. According to a further embodiment, the liposomes comprise about 2 mol % to about 10 mol %, or about 4 mol % to about 8 mol %, or about 5 mol % to about 7 mol % of MPEG-DSPE.

According to some embodiments, the liposomes according to the present invention comprise distearoyl, i.e. lipopolymers comprising stearoyl moieties. Examples of such lipopolymers are distearoylglycerol and polyethyleneglycol oxycarbonyl-3-amino-1,2-propanediol distearoylester. Such lipopolymers may be PEGylated. Thus in one embodiment, liposomes of the present invention comprise MPEG-DSG, MPEG-DS or a combination thereof. According to some embodiments, PEG of the MPEG-DSPE has an average molecular weight of about 350 Da to about 100,000 Da, about 500 Da to about 40,000 Da, about 750 Da to about 20,000 Da, about 1,000 Da to about 12,000 Da, or about 1500 Da to about 5,000 Da or about 2000 Da to about 4,000 Da.

According to some embodiments, the liposomes of the present invention comprise phosphatidylcholine and DSPE. According to some embodiments, at least one of said phosphatidylcholine and DSPE is pegylated. According to some embodiments, the liposome comprises HSPC and DSPE. According to other embodiments, the liposomes comprises phosphatidylcholine and MPEG-DSPE. According to a further embodiment, the liposomes comprise HSPC and MPEG-DSPE.

According to any one of the above embodiment, the liposomes of the present invention are substantially devoid of cholesterol.

According to any one of the above embodiments, the liposomes according to the present invention further comprise cholesterol. According to some embodiment, the liposomes comprise about 0 mol % to about 50 mol %, about 10 mol % to about 40 mol %, about 20 mol % to about 30 mol % cholesterol. According to some embodiments, the composition comprises about 30 mol % to about 50 mol %, about 35 mol % to about 45 mol % or about 38 mol % to about 42 mol % of cholesterol. According to further embodiments, the liposomes comprise about 1 mol % to about 10 mol %, about 2 mol % to about 8 mol %, about 3 mol % to about 7 mol %, about 4 mol % to about 6 mol %, or about 4 mol % to about 8 mol % of cholesterol.

Lipids may also contain lipid protective agents, such as, but not limited to, α-tocopherol, α-tocopherol acetate, or α-tocopherol succinate to protect the lipid components against free radical damage. Typically such agents are included at a mole percentage in the range of 0.5-2 mol %. In certain instances it is advantageous to add α-tocopherol to the liposomes to maintain a balance between vitamin E and polysaturated lipids in the liposomes.

According to any one of the above embodiments, the liposomes according to the present invention have the average diameter of about 10 nm to about 300 nm, about 20 nm to about 200 nm, or about 50 nm to about 100 nm. According to some embodiments, the liposomes according to the present invention have the average diameter of about 10 nm to about 100 nm, 20 nm to about 90 nm, 30 nm to about 80 nm, 40 nm to about 70 nm, or about 50 nm to about 60 nm. According to one embodiment, the liposomes according to the present invention have the average diameter of about 60 nm to about 90 nm. According to another the liposomes according to the present invention have the average diameter of about 70 nm to about 85 nm. According to yet another embodiment, the liposomes according to the present invention have the average diameter less than 150 nm or less than 100 nm.

Liposomes according to the present invention can be prepared by any techniques known to those knowledgeable in the art. Different types of liposomes, and in particular those having an average diameter size of less than 300 nm, less than 200 nm or less than 100 nm may be employed in the context of the present invention.

According to any one of the above embodiments, the liposomes according to the present invention and the pharmaceutical composition comprising said liposome are substantially devoid of any compound being an active agent prescribed for treatment of any autoimmune disease. According to any one of the above embodiments, the liposomes according to the present invention and the pharmaceutical composition comprising said liposome substantially devoid of any compound being an active agent used for treatment of any autoimmune disease. According to any one of the above embodiments, the liposomes according to the present invention and the pharmaceutical composition comprising said liposome substantially devoid of any compound being an active agent prescribed for treatment of a gastrointestinal autoimmune disease. According to another embodiment, the liposomes according to the present invention and the pharmaceutical composition comprising said liposome substantially devoid of steroids, and in particularly corticosteroids. According to yet another embodiment, the liposomes according to the present invention and the pharmaceutical composition comprising said liposome substantially devoid of an anti-inflammatory agent, and in particular a non-steroidal anti-inflammatory agent. According to a further embodiment, the liposomes, according to the present invention, and the pharmaceutical composition comprising said liposomes are substantially devoid of compounds being an active agent in a medicament used or prescribed for treatment of IBD. According to some embodiments, the liposomes or compositions according to the invention are devoid of an active agent selected from aminosalicylates such as mesalamine (5-aminosalicylic acid), olsalazine, baslalazide or sulfasalazine, immunomodulators such as azathioprine, 6-mercaptopurine or methotrexate, anti-cytokine drugs such as infliximab, adalimumab, certolizumab pegol, golimumab or ustekinumab, anti-cell adhesion molecules such as vedolizumab or natalizumab, and any combination thereof. According to some embodiments, the term “prescribed” has the meaning of “used”. According to a further embodiment, the liposomes, according to the present invention, and the pharmaceutical composition comprising said liposomes are substantially devoid of compounds being an active agent in a prescription drug.

According to some embodiments, said liposomes are a drug-free liposome. The term “drug free” refers to an “empty” liposome or vesicle lacking any active ingredient of a pharmaceutical drug, as known in the art.

According to any one of the above embodiments, the liposomes of the present invention comprise one or more non-active compounds. According to some embodiments, the non-active compound is selected from minerals such as Ca, Mg, Mn, Ir, Se, P, K, Na, and Zn, salts, vitamins such as Vitamin A, B1, B2, B3, B5, B6, B7, B12, C, D, E, K, and folic acid, buffers, antioxidants, nutraceuticals, food additives and supplements, and pharmaceutically acceptable excipients.

According to any one of the above embodiments, the pharmaceutical composition comprising the liposomes according to the present invention are administered orally, rectally or parenterally.

According to the teachings of the present invention, the method comprises administering of a pharmaceutical composition comprising a therapeutically effective amount of liposomes of the present invention. According to some embodiments, the pharmaceutical composition comprises about 10 to about 200 mg/ml of the empty liposomes of the present invention. According to certain embodiments, the pharmaceutical composition comprises about 15 to about 190, about 20 to about 180, about 30 to about 170, about 40 to about 160, about 50 to about 150, about 60 to about 140, about 70 to about 130, about 80 to about 120, or about 90 to about 110 mg/ml. According to one particular embodiment, the pharmaceutical composition comprises about 40 to about 120 mg/ml of liposomes, or about 50 to about 110 mg/ml of liposomes. According to another embodiment, the pharmaceutical composition comprises about 35 to about 45 mg/ml of liposomes. According to some embodiments, the liposomes are dried liposomes. In certain embodiment, the liposomes are suspended in a liquid carrier, e.g. aqueous carrier, prior use.

According to some embodiments, the liposomes comprise phosphatidylcholine such as HSPC and cholesterol. According to one embodiment, the liposomes comprise about 40 mol % to about 80 mol % phosphatidylcholine such as HSPC, and about 20 mol % to about 60 mol % cholesterol. According to another embodiment, the liposomes comprises about 50 mol % to about 70 mol % phosphatidylcholine such as HSPC, and about 30 mol % to about 50 mol % cholesterol. According to a further embodiment, the liposomes comprises about 55 mol % to about 65 mol % phosphatidylcholine such as HSPC, and about 35 mol % to about 45 mol % cholesterol. In yet another embodiment, the liposomes comprises about 61 mol % HSPC, and 36 mol % cholesterol.

According to some embodiments, the liposomes according to the present invention comprise phosphatidylcholine such as HSPC, cholesterol and/or DSPE, such as MPEG-DSPE. According to some embodiments, the liposomes according to the present invention comprise phosphatidylcholine, such as HSPC, and DSPE such as MPEG-DSPE. According to some embodiments, the liposomes comprise about 30 mol % to about 100 mol % phosphatidylcholine such as HSPC, and about 0 mol % to about 30 mol % DSPE such as MPEG-DSPE. According to any one of the above embodiments, the MPEG of the MPEG-DSPE has an average molecular weight of about 350 Da to about 100,000 Da, about 500 Da to about 40,000 Da, about 750 Da to about 20,000 Da, about 1,000 Da to about 12,000 Da, or about 1500 Da to about 5,000 Da or about 2000 Da to about 4,000 Da, or about 2000 Da. According to some embodiment, the liposomes are substantially devoid of cholesterol. According to other embodiments, the liposomes according to the present invention comprise phosphatidylcholine, and in particular HSPC, cholesterol and DSPE, such as MPEG-DSPE. According to some embodiments, the liposomes comprise about 30 mol % to about 100 mol % phosphatidylcholine such as HSPC, about 0 mol % to about 50 mol % cholesterol and about 0 mol % to about 20 mol % DSPE such as MPEG-DSPE. According to some more particular embodiments, the liposome comprises about 40 mol % to about 70 mol % HSPC, about 30 mol % to about 50 mol % cholesterol and about 2 mol % to about 10 mol % MPEG-DSPE. According to some particular embodiments, the liposomes comprise about 50 mol % to about 60 mol % HSPC, about 35 mol % to about 45 mol % cholesterol and about 4 mol % to about 8 mol % MPEG-DSPE. According to another particular embodiment, the liposomes comprise about 55 mol % HSPC, about 39 mol % cholesterol and about 6 mol % MPEG-DSPE. According to any one of the above embodiments, the MPEG of the MPEG-DSPE has an average molecular weight of about 350 Da to about 100,000 Da, about 500 Da to about 40,000 Da, about 750 Da to about 20,000 Da, about 1,000 Da to about 12,000 Da, or about 1500 Da to about 5,000 Da or about 2000 Da to about 4,000 Da. In one particular embodiment, the MPEG has a molecular weight of 2000 Da. According to some embodiments, such liposomes have an average diameter of about 10 nm to about 300 nm, about 20 nm to about 200 nm, or about 50 nm to about 100 nm. According to some embodiments, the liposomes according to the present invention have the average diameter of about 10 nm to about 100 nm, 20 nm to about 90 nm, 30 nm to about 80 nm, 40 nm to about 70 nm, or about 50 nm to about 60 nm. According to one embodiment, the liposomes according to the present invention have the average diameter of 60 nm to about 90 nm. According to another embodiment, the liposomes according to the present invention have the average diameter of 70 nm to about 85 nm. According to some embodiments, the liposomes are substantially devoid of a compound selected from a compound prescribed for treatment of a gastrointestinal autoimmune disease, a compound prescribed for treatment of an autoimmune disease, a compound prescribed for treatment of IBD, an active agent in a prescription drug, steroids, anti-inflammatory compound or drug, or non-steroidal anti-inflammatory compound or drug. In particular such liposomes are substantially devoid of a compound selected from aminosalicylates such as mesalamine (5-aminosalicylic acid), olsalazine, baslalazide or sulfasalazine, immunomodulators such as azathioprine, 6-mercaptopurine or methotrexate, anti-cytokine drugs such as infliximab, adalimumab, certolizumab pegol, golimumab or ustekinumab, anti-cell adhesion molecules such as vedolizumab or natalizumab. In one more particular embodiment, the liposomes are drug-free liposomes. According to some embodiments, the liposomes comprise one or more non-active compounds. According to some embodiments, the non-active compound is selected from minerals such as Ca, Mg, Mn, Ir, Se, P, K, Na, and Zn, salts, vitamins such as Vitamin A, B1, B2, B3, B5, B6, B7, B12, C, D, E, K, folic acid, buffers, antioxidants, nutraceuticals, food additives and supplements, and pharmaceutically acceptable excipients. According to any one of the above embodiments, the liposomes and/or composition is administered orally, rectally or parenterally. According to some embodiments, the pharmaceutical composition comprises about 10 to about 200 mg/ml of the empty liposomes of the present invention. According to certain embodiments, the pharmaceutical composition comprises about 15 to about 190, about 20 to about 180, about 30 to about 170, about 40 to about 160, about 50 to about 150, about 60 to about 140, about 70 to about 130, about 80 to about 120, or about 90 to about 110 mg/ml. According to one particular embodiment, the pharmaceutical composition comprises about 40 to about 120 mg/ml of liposomes, or about 50 to about 110 mg/ml of liposomes. According to another embodiment, the pharmaceutical composition comprises about 35 to about 45 mg/ml of liposomes. According to some embodiments, the liposomes are dried liposomes. In certain embodiment, the liposomes are suspended in a liquid carrier, e.g. aqueous carrier, prior use.

According to some embodiments, the present invention provides a method for treatment of inflammatory bowel disease such as Crohn's disease or ulcerative colitis said method comprising administering a pharmaceutically acceptable composition comprising a therapeutically effective amount of SUV liposomes, wherein said composition is substantially devoid of a compound being an active agent in a medicament prescribed for treatment of autoimmune disease, gastrointestinal autoimmune disease or IBD. According to some embodiments, the liposomes are SUV liposomes comprising about 30 mol % to about 100 mol % phosphatidylcholine such as HSPC, and about 0 mol % to about 30 mol % DSPE such as MPEG-DSPE, wherein the PEG has the average molecular weight of 2000 Da. According to further embodiments, the liposomes are SUV liposomes comprising about 30 mol % to about 100 mol % phosphatidylcholine such as HSPC, and about 0 mol % to about 30 mol % DSPE such as MPEG-DSPE, wherein the PEG has the average molecular weight of 2000 Da. According to other embodiments, the liposomes are SUV liposomes comprising about 50 mol % to about 60 mol % HSPC, about 30 mol % to about 50 mol % cholesterol and about 3 mol % to about 9 mol % MPEG-DSPE, wherein the PEG has the average molecular weight of 2000 Da, and the diameter of said liposomes is about 70 to about 85 nm. According to other embodiments, the liposomes are SUV liposomes comprising 55 mol % HSPC, 39 mol % cholesterol and 6 mol % MPEG-DSPE, wherein the PEG have the average molecular weight of 2000 Dalton, and the average diameter of said liposomes is about 78 nm. According yet another embodiment, the liposomes are SUV liposomes comprising about 55 mol % to about 65 mol % phosphatidylcholine such as HSPC, and about 35 mol % to about 45 mol % cholesterol, wherein the diameter of said liposomes is about 70 to about 85 nm. In still another embodiment, the liposomes comprises about 61 mol % HSPC, and 36 mol % cholesterol. The liposomes defined hereinabove and pharmaceutically acceptable composition comprising said liposomes are substantially devoid of a compound selected from a compound being an active agent in a medicament used or prescribed for treatment of autoimmune disease, gastrointestinal autoimmune disease or IBD; an active agent of a prescription drug; steroid; anti-inflammatory compound or drug; or non-steroidal anti-inflammatory compound or drug. In some particular embodiments, the liposomes and composition comprising said liposomes are substantially devoid of a compound selected from aminosalicylates such as mesalamine (5-aminosalicylic acid), olsalazine, baslalazide or sulfasalazine, immunomodulators such as azathioprine, 6-mercaptopurine or methotrexate, anti-cytokine drugs such as infliximab, adalimumab, certolizumab pegol, golimumab or ustekinumab, anti-cell adhesion molecules such as vedolizumab or natalizumab. In one more particular embodiment, the liposomes are drug-free liposomes. According to some embodiments, the liposome comprise one or more non-active compounds such as minerals, salts, vitamins, buffers, antioxidants, nutraceuticals, food additives and supplements, and pharmaceutically acceptable excipients. According to one embodiment, the composition comprising said liposomes is administered orally, rectally or parenterally. The term “comprising” as used herein comprises the term “consisting”. Thus, according to some embodiments, the liposomes of the present invention consist the components as defined hereinabove. According to some embodiments, the liposomes are SUV liposomes consisting about 50 mol % to about 60 mol % HSPC, about 30 mol % to about 50 mol % cholesterol and about 3 mol % to about 9 mol % MPEG-DSPE, wherein the PEG has the average molecular weight of 2000 Da. According to another embodiment, the liposomes are SUV liposomes consisting of 55 mol % HSPC, 39 mol % cholesterol and 6 mol % MPEG-DSPE, wherein the PEG have the average molecular weight of 2000 Dalton, the and the average diameter of said liposomes is about 78 nm. According to yet another embodiment, the liposomes are SUV liposomes consisting of 55 mol % to about 65 mol % phosphatidylcholine such as HSPC, and about 35 mol % to about 45 mol % cholesterol, wherein the diameter of said liposomes is about 70 to about 85 nm. According to some embodiments, the pharmaceutical composition comprises about 10 to about 200 mg/ml of the empty liposomes of the present invention. According to certain embodiments, the pharmaceutical composition comprises about 15 to about 190, about 20 to about 180, about 30 to about 170, about 40 to about 160, about 50 to about 150, about 60 to about 140, about 70 to about 130, about 80 to about 120, or about 90 to about 110 mg/ml. According to one particular embodiment, the pharmaceutical composition comprises about 40 to about 120 mg/ml of liposomes, or about 50 to about 110 mg/ml of liposomes. According to another embodiment, the pharmaceutical composition comprises about 35 to about 45 mg/ml of liposomes. According to some embodiments, the liposomes are dried liposomes. In certain embodiment, the liposomes are suspended in a liquid carrier, e.g. aqueous carrier, prior use.

According to any one of the above embodiment, the liposome of the present invention comprise a liquid hydrophilic water-based phase entrapped within the liposome. According to some embodiment, said hydrophilic phase comprise a buffer. According to some embodiments, the hydrophilic phase comprise about 5% to about 15% sucrose and about 5 mM to about 15 mM histidine, pH 6.5.

According to another aspect, the present invention provides a pharmaceutical composition comprising small unilamellar vesicle (SUV) liposomes for use in treatment of an autoimmune disease, wherein said composition is substantially devoid of a compound being an active agent in a medication prescribed for treatment of an autoimmune disease. The liposomes and the compositions are as disclosed above. Thus in one embodiment, the present invention provides a composition comprising SUV liposomes for use in treatment of an autoimmune disease, with a proviso that composition and said liposomes are substantially devoid of a compound being an active agent in a medication prescribed for treatment of an autoimmune disease. The autoimmune disease that may be treated by the composition is as described above, e.g. a gastrointestinal autoimmune disease. Thus in one embodiment, the present invention provides a pharmaceutical composition comprising small unilamellar vesicle (SUV) liposomes for use in treatment of a gastrointestinal autoimmune disease, wherein said composition is substantially devoid of a compound being an active agent in a medication prescribed for treatment of an gastrointestinal autoimmune disease. According to some embodiment, the composition is substantially devoid of a compound being an active agent in a medication prescribed for treatment of autoimmune disease. According to some embodiment, the gastrointestinal autoimmune disease is as defined herein above. In one embodiment, the gastrointestinal autoimmune disease is selected from IBD and IBS. In some embodiments the gastrointestinal autoimmune disease is selected from ulcerative colitis, Crohn's disease, celiac disease, irritable bowel syndrome or autoimmune hepatitis. In some embodiments the autoimmune disorder is other than rheumatoid arthritis. According to one particular embodiment, an autoimmune disease is Crohn's disease, ulcerative colitis or IBS. According to some embodiments, the term “prescribed” has the meaning of “used”. According to some embodiments, the liposomes of the present invention comprise liposome-forming lipids such as phosphatidylcholine and lipopolymers. According to some embodiment, the phosphatidylcholine is e.g. egg yolk phosphatidylcholine (EPC), 1-palmitoyl-2-oleoylphosphatidyl choline (POPC), hydrogenated soy phosphatidylcholine (HSPC), distearoylphosphatidylcholine (DSPC) or dimyristoyl phosphatidylcholine (DMPC). In some embodiments, the liposomes comprise about 30 mol % to about 100 mol %, about 40 mol % to about 90 mol %, about 50 mol % to about 80 mol %, or about 60 mol % to about 70 mol % of phosphatidylcholine. According to more particular embodiments, the liposome comprise hydrogenated soy phosphatidylcholine (HSPC), e.g. about 30 mol % to about 100 mol %, about 40 mol % to about 90 mol %, about 50 mol % to about 80 mol %, or about 60 mol % to about 70 mol % of HSPC.

According to any one of the above embodiments, the liposomes of the present invention comprise stabilizing polymer molecules. According to one embodiment, the polymer is selected from polyalkylether, polysialic acid, polylactic acid and polyglycolic acid. According to one embodiment, polyalkylether is polyethylene glycol. Therefore according to some embodiments, the liposome according to the present invention is PEGylated liposome, i.e. a liposome comprising PEG molecules. According to one embodiment, the PEG moiety attached to the liposomes according to the present invention has an average molecular weight of about 350 Da to about 100,000 Da, about 500 Da to about 40,000 Da, about 750 Da to about 20,000 Da, about 1,000 Da to about 12,000 Da, or about 1500 Da to about 5,000 Da or about 2000 Da to about 4,000 Da. According to a further embodiment, the PEG moiety has an average molecular weight of about 1500 to about 3000 Da.

According to some embodiments, the liposomes comprise phosphatidylethanolamine such as distearylphosphatidylethanolamine (DSPE). According to some embodiments, phosphatidylethanolamine is PEGylated phosphatidylethanolamine, i.e. phosphatidylethanolamine conjugated with PEG molecules. According one embodiment, the liposomes comprise PEGylated DSPE, in particular methyl-polyethyleneglycol distearylphosphatidylethanolamine (MPEG-DSPE). According to some embodiments, methyl-polyethyleneglycol (MPEG) of the MPEG-DSPE has an average molecular weight of about 350 Da to about 100,000 Da, about 500 Da to about 40,000 Da, about 750 Da to about 20,000 Da, about 1,000 Da to about 12,000 Da, or about 1500 Da to about 5,000 Da or about 2000 Da to about 4,000 Da. According to a further embodiment, the PEG moiety has an average molecular weight of about 1500 to about 3000 Da. According to some embodiments, the liposome comprise about 0 mol %, e.g. 0.001 mol %, 0.01 mol % or 0.1 mol % to about 20 mol %, about 5 mol % to about 15 mol %, or about 10 mol % of MPEG-DSPE According to another embodiment, the liposome comprise about 0.5 mol % to about 18 mol %, about 1 mol % to about 16 mol %, about 3 mol % to about 15 mol %, about 5 mol % to about 14 mol %, about 7 mol % to about 13 mol %, about 8 mol % to about 12 mol %, or about 10 mol % of MPEG-DSPE. According to a further embodiment, the liposomes comprise about 2 mol % to about 10 mol %, or about 4 mol % to about 8 mol %, or about 5 mol % to about 7 mol % of MPEG-DSPE.

According to some embodiments, the liposomes according to the present invention comprise distearoyl, i.e. lipopolymers comprising two stearoyl moieties. Examples of such lipopolymers are distearoylglycerol and polyethylene glycol oxycarbonyl-3-amino-1,2-propanediol distearoylester. Such lipopolymers may be PEGylated. Thus in one embodiment, liposomes of the present invention comprise MPEG-DSG, MPEG-DS or a combination thereof. According to some embodiments, PEG of the MPEG-DSPE has an average molecular weight of about 350 Da to about 100,000 Da, about 500 Da to about 40,000 Da, about 750 Da to about 20,000 Da, about 1,000 Da to about 12,000 Da, or about 1500 Da to about 5,000 Da or about 2000 Da to about 4,000 Da.

According to any one of the above embodiment, the liposomes of the present invention are substantially devoid of cholesterol.

According to any one of the above embodiments, the liposomes further comprise cholesterol. According to some embodiment, the liposomes comprise about 0 mol % to about 50 mol %, about 10 mol % to about 40 mol %, about 20 mol % to about 30 mol % cholesterol. According to some embodiments, the composition comprises about 30 mol % to about 50 mol %, about 35 mol % to about 45 mol % or about 38 mol % to about 42 mol % of cholesterol. According to further embodiments, the liposomes comprise about 1 mol % to about 10 mol %, about 2 mol % to about 8 mol %, about 3 mol % to about 7 mol %, about 4 mol % to about 6 mol %, or about 4 mol % to about 8 mol % of cholesterol.

According to any one of the above embodiments, the liposomes according to the present invention have the average diameter of about 10 nm to about 300 nm, about 20 nm to about 200 nm, or about 50 nm to about 100 nm. According to some embodiments, the liposomes according to the present invention have the average diameter of about 10 nm to about 100 nm, 20 nm to about 90 nm, 30 nm to about 80 nm, 40 nm to about 70 nm, or about 50 nm to about 60 nm. According to one embodiment, the liposomes according to the present invention have the average diameter of about 60 to about 90 nm. According to another embodiment, the liposomes according to the present invention have the average diameter of about 70 nm to about 85 nm. According to yet another embodiment, the liposomes according to the present invention have the average diameter less than 150 nm or less than 100 nm.

According to any one of the above embodiments, the liposomes according to the present invention and the pharmaceutical composition comprising said liposomes are substantially devoid of any compound being an active agent prescribed for treatment of any autoimmune disease. According to another embodiment, the liposomes according to the present invention and the pharmaceutical composition comprising said liposomes are substantially devoid of a compound being an active agent prescribed for treatment of a gastrointestinal autoimmune disease. According to another embodiment, the liposomes according to the present invention and the pharmaceutical composition comprising said liposomes are substantially devoid of steroids, and in particularly corticosteroids. According to yet another embodiment, the liposomes according to the present invention and the pharmaceutical composition comprising said liposomes are substantially devoid of an inti-inflammatory agent, and in particularly a non-steroidal inti-inflammatory agent. According to a further embodiment, the liposomes, according to the present invention, and the pharmaceutical composition comprising said liposomes are substantially devoid of compound being an active agent in a medicament used or prescribed for treatment of IBD. According to some embodiments, said active agent is selected from aminosalicylates such as mesalamine (5-aminosalicylic acid), olsalazine, baslalazide or sulfasalazine, immunomodulators such as azathioprine, 6-mercaptopurine or methotrexate, anti-cytokine drugs such as infliximab, adalimumab, certolizumab pegol, golimumab or ustekinumab, anti-cell adhesion molecules such as vedolizumab or natalizumab, and any combination thereof. According to a further embodiment, the liposomes, according to the present invention, and the pharmaceutical composition comprising said liposomes are substantially devoid of compounds being an active agent in a prescription drug.

According to some embodiments, said liposomes are a drug-free liposomes.

According to any one of the above embodiments, the liposomes of the present invention comprise one or more non-active compound. According to some embodiments, the non-active compound is selected from minerals such as Ca, Mg, Mn, Ir, Se, P, K, Na, and Zn, salts, vitamins such as Vitamin A, B1, B2, B3, B5, B6, B7, B12, C, D, E, K, folic acid, buffers, antioxidants, nutraceuticals, food additives and supplements, and pharmaceutically acceptable excipients.

According to any one of the above embodiments, the pharmaceutical composition comprising the liposomes according to the present invention is administered orally, rectally or parenterally.

According to some embodiments, the pharmaceutical composition comprises about 10 to about 200 mg/ml of the empty liposomes of the present invention. According to certain embodiments, the pharmaceutical composition comprises about 15 to about 190, about 20 to about 180, about 30 to about 170, about 40 to about 160, about 50 to about 150, about 60 to about 140, about 70 to about 130, about 80 to about 120, or about 90 to about 110 mg/ml. According to one particular embodiment, the pharmaceutical composition comprises about 40 to about 120 mg/ml of liposomes, or about 50 to about 110 mg/ml of liposomes. According to another embodiment, the pharmaceutical composition comprises about 35 to about 45 mg/ml of liposomes. According to some embodiments, the liposomes are dried liposomes. In certain embodiment, the liposomes are suspended in a liquid carrier, e.g. aqueous carrier, prior use.

According to some particular embodiments, the present invention provides pharmaceutical composition comprising SUV liposomes as defined hereinabove for use in treatment of an autoimmune disease. According to some particular embodiments, the present invention provides pharmaceutical composition comprising SUV liposomes as defined hereinabove for use in treatment of a gastrointestinal autoimmune disease. According to some embodiments, the liposomes comprise phosphatidylcholine, e.g. HSPC, and may further comprise DSPE, such as MPEG-DSPE. According to some embodiments, the liposomes are substantially devoid of cholesterol. According to one particular embodiments, the liposomes according to the present invention comprise phosphatidylcholine such as HSPC and further comprise cholesterol. According to another embodiment, the liposomes comprise about 30 mol % to about 100 mol % phosphatidylcholine such as HSPC, and about 0 mol % to about 50 mol % cholesterol. According to further particular embodiments, the liposomes according to the present invention comprise phosphatidylcholine, such as HSPC, and DSPE such as MPEG-DSPE. According to some embodiments, the liposomes comprise about 30 mol % to about 100 mol % phosphatidylcholine such as HSPC, and about 0 mol % to about 30 mol % DSPE such as MPEG-DSPE. According to some embodiments, the MPEG of the MPEG-DSPE has an average molecular weight of about 350 Da to about 100,000 Da, about 500 Da to about 40,000 Da, about 750 Da to about 20,000 Da, about 1,000 Da to about 12,000 Da, or about 1500 Da to about 5,000 Da or about 2000 Da to about 4,000 Da, or about 2000 Da. According to a further particular embodiments, the liposomes according to the present invention comprise phosphatidylcholine, and in particular HSPC, cholesterol and DSPE, such as MPEG-DSPE. According to some embodiments, the liposomes comprise about 30 mol % to about 100 mol % phosphatidylcholine such as HSPC, about 0 mol % to about 50 mol % cholesterol and about 0 mol % to about 20 mol % DSPE such as MPEG-DSPE. According to some more particular embodiments, the liposome comprises about 40 mol % to about 70 mol % HSPC, about 30 mol % to about 50 mol % cholesterol and about 2 mol % to about 10 mol % MPEG-DSPE. According to some particular embodiments, the liposomes comprise about 50 mol % to about 60 mol % HSPC, about 35 mol % to about 45 mol % cholesterol and about 4 mol % to about 8 mol % MPEG-DSPE. According to another particular embodiment, the liposomes comprise about 55 mol % HSPC, about 39 mol % cholesterol and about 6 mol % MPEG-DSPE. According to any one of the above embodiments, the MPEG of the MPEG-DSPE has an average molecular weight of about 350 Da to about 100,000 Da, about 500 Da to about 40,000 Da, about 750 Da to about 20,000 Da, about 1,000 Da to about 12,000 Da, about 1500 Da to about 5,000 Da or about 2000 Da to about 3,000 Da. In one particular embodiment, the MPEG has a molecular weight of 2000 Da. According to other embodiments, the liposomes are SUV liposomes comprising about 55 mol % to about 65 mol % phosphatidylcholine such as HSPC, and about 35 mol % to about 45 mol % cholesterol. In yet another embodiment, the liposomes comprises about 61 mol % HSPC, and 36 mol % cholesterol. According to some embodiments, such liposomes have an average diameter of about 10 nm to about 300 nm, about 20 nm to about 200 nm, or about 50 nm to about 100 nm. According to some embodiments, the liposomes according to the present invention have the average diameter of about 10 nm to about 100 nm, 20 nm to about 90 nm, 30 nm to about 80 nm, 40 nm to about 70 nm, or about 50 nm to about 60 nm. According to one embodiment, the liposomes according to the present invention have the average diameter of 60 nm to about 90 nm. According to another embodiment, the liposomes according to the present invention have the average diameter of 70 nm to about 85 nm According to some embodiments, the liposomes are substantially devoid of a compound selected from a compound prescribed for treatment of autoimmune disease, a compound prescribed for treatment of gastrointestinal autoimmune disease, a compound prescribed for treatment of IBD, active agent in a prescription drug, steroid, anti-inflammatory compound or drug, and non-steroidal anti-inflammatory compound or drug. In particular embodiments, the liposomes are substantially devoid of a compound selected from aminosalicylates such as mesalamine (5-aminosalicylic acid), olsalazine, baslalazide or sulfasalazine, immunomodulators such as azathioprine, 6-mercaptopurine or methotrexate, anti-cytokine drugs such as infliximab, adalimumab, certolizumab pegol, golimumab or ustekinumab, anti-cell adhesion molecules such as vedolizumab or natalizumab. In one more particular embodiment, the liposomes are drug-free liposomes. According to some embodiments, the liposomes of the present invention comprise one or more non-active compounds. According to some embodiments, the non-active compound is selected from minerals such as Ca, Mg, Mn, Ir, Se, P, K, Na, and Zn, salts, vitamins such as Vitamin A, B1, B2, B3, B5, B6, B12, C, D, E, K, folic acid, buffers, antioxidants, nutraceuticals, food additives and supplements, and pharmaceutically acceptable excipients. According to any one of the above embodiments, the liposomes and/or composition are administered orally, rectally or parenterally. According to some embodiments, the pharmaceutical composition comprises about 10 to about 200 mg/ml of the empty liposomes of the present invention. According to certain embodiments, the pharmaceutical composition comprises about 15 to about 190, about 20 to about 180, about 30 to about 170, about 40 to about 160, about 50 to about 150, about 60 to about 140, about 70 to about 130, about 80 to about 120, or about 90 to about 110 mg/ml. According to one particular embodiment, the pharmaceutical composition comprises about 40 to about 120 mg/ml of liposomes, or about 50 to about 110 mg/ml of liposomes. According to another embodiment, the pharmaceutical composition comprises about 35 to about 45 mg/ml of liposomes. According to some embodiments, the liposomes are dried liposomes. In certain embodiment, the liposomes are suspended in a liquid carrier, e.g. aqueous carrier, prior use.

According to some embodiments, the present invention provides a pharmaceutically acceptable composition comprising SUV liposomes, for use in treatment of IBD disease such as Crohn's disease or ulcerative colitis, wherein said composition is substantially devoid of a compound being an active agent in a medication prescribed for treatment of an autoimmune disease, gastrointestinal autoimmune disease or IBD disease. According to some embodiments, the liposomes are SUV liposomes comprising about 30 mol % to about 100 mol % phosphatidylcholine such as HSPC, and about 0 mol % to about 30 mol % DSPE such as MPEG-DSPE, wherein the PEG has the average molecular weight of 2000 Da. According to some embodiments, the liposomes are SUV liposomes comprising about 50 mol % to about 60 mol % HSPC, about 30 mol % to about 50 mol % cholesterol and about 3 mol % to about 9 mol % MPEG-DSPE, wherein the PEG has the average molecular weight of 2000 Da, the diameter of said liposomes is about 70 to about 85 nm. According to another embodiment, the liposomes are SUV liposomes comprising about 55 mol % to about 65 mol % phosphatidylcholine such as HSPC, and about 35 mol % to about 45 mol % cholesterol, wherein the diameter of said liposomes is about 70 to about 85 nm. In yet another embodiment, the liposomes comprises about 61 mol % HSPC, and 36 mol % cholesterol. The liposomes as defined hereinabove and composition comprising said liposomes are substantially devoid of a compound selected from a compound used or prescribed for treatment of GI autoimmune disease or IBD; an active agent in a prescription drug; steroid; anti-inflammatory compound or drug; or non-steroidal anti-inflammatory compound or drug. In particular embodiments, the liposomes and composition comprising said liposomes are substantially devoid of a compound selected from aminosalicylates such as mesalamine (5-aminosalicylic acid), olsalazine, baslalazide or sulfasalazine, immunomodulators such as azathioprine, 6-mercaptopurine or methotrexate, anti-cytokine drugs such as infliximab, adalimumab, certolizumab pegol, golimumab or ustekinumab, anti-cell adhesion molecules such as vedolizumab or natalizumab. In one more particular embodiment, the liposomes are drug-free liposomes. According to some embodiments, the liposomes comprise one or more non-active compounds such minerals, salts, vitamins, buffers, antioxidants, nutraceuticals, food additives and supplements, and pharmaceutically acceptable excipients. According to one embodiment, the liposomes and/or the composition comprising said liposomes are administered orally, rectally or parenterally. According to some embodiments, the liposomes are SUV liposomes comprising about 30 mol % to about 100 mol % of HSPC, and about 0 mol % to about 30 mol % of MPEG-DSPE, wherein the MPEG has the average molecular weight of 2000 Da. According to other embodiments, the liposomes are SUV liposomes comprising 55 mol % HSPC, 39 mol % cholesterol and 6 mol % MPEG-DSPE, wherein the PEG have the average molecular weight of 2000 Dalton, and the average diameter of said liposomes is about 78 nm. According to one embodiment, the liposomes and/or composition comprising said liposomes are administered orally, rectally or parenterally. According to some embodiments, the pharmaceutical composition comprises about 10 to about 200 mg/ml of the empty liposomes of the present invention. According to certain embodiments, the pharmaceutical composition comprises about 15 to about 190, about 20 to about 180, about 30 to about 170, about 40 to about 160, about 50 to about 150, about 60 to about 140, about 70 to about 130, about 80 to about 120, or about 90 to about 110 mg/ml. According to one particular embodiment, the pharmaceutical composition comprises about 40 to about 120 mg/ml of liposomes, or about 50 to about 110 mg/ml of liposomes. According to another embodiment, the pharmaceutical composition comprises about 35 to about 45 mg/ml of liposomes.

The term “comprising” as used herein comprises the term “consisting”. Thus according to some embodiments, the liposomes of the present invention consist of the components as defined hereinabove. According to some embodiments, the liposomes are SUV liposomes comprising about 30 mol % to about 100 mol % phosphatidylcholine such as HSPC, and about 0 mol % to about 30 mol % DSPE such as MPEG-DSPE, wherein the PEG has the average molecular weight of 2000 Da. According to some embodiments, the liposomes are SUV liposomes consisting of about 50 mol % to about 60 mol % HSPC, about 30 mol % to about 50 mol % cholesterol and about 3 mol % to about 9 mol % MPEG-DSPE, wherein the PEG has the average molecular weight of 2000 Da, and the wherein the average diameter of said liposomes is about 70 to about 85 nm. According to another embodiment, the liposomes are SUV liposomes consisting of 55 mol % HSPC, 39 mol % cholesterol and 6 mol % MPEG-DSPE, wherein the PEG have the average molecular weight of 2000 Dalton, wherein the average diameter of said liposomes is about 70 to about 85 nm of said liposomes is about 78 nm. According to yet another embodiment, the liposomes are SUV liposomes consisting of 55 mol % to about 65 mol % phosphatidylcholine such as HSPC, and about 35 mol % to about 45 mol % cholesterol, wherein the average diameter of said liposomes is about 70 to about 85 nm. According to one embodiment, the liposomes consist of HSPC and have the diameter of about 70 to about 85 nm

According to any one of the above embodiment, the liposome of the present invention comprise a liquid hydrophilic water-based phase entrapped within the liposome. According to some embodiment, said hydrophilic phase comprise a buffer. According to some embodiments, the hydrophilic phase comprise about 5% to about 15% sucrose and about 5 mM to about 15 mM histidine, pH 6.5.

According to another aspect the present invention provides a use of SUV liposomes for preparing a medicament for treating of autoimmune disease, wherein said composition and liposomes are substantially devoid of a compound being an active agent in a medication prescribed for treatment of an autoimmune disease. According to one embodiment, the medicament is for treating a gastrointestinal autoimmune disease, wherein said composition and liposomes are substantially devoid of a compound being an active agent in a medication prescribed for treatment of a gastrointestinal autoimmune disease. According to another embodiment, the medicament is for treating a gastrointestinal autoimmune disease, wherein said composition and liposomes are substantially devoid of a compound being an active agent in a medication prescribed for treatment of an autoimmune disease.

The term “comprising” as used herein comprises the term “consisting”.

As used herein, the term “about”, when referring to a measurable value such as an amount, a temporal duration, and the like, is meant to encompass variations of +/−10%, or +/−5%, +/−1%, or even +/−0.1% from the specified value.

According to some embodiments, the term “about 0 mol %” refer to 0.0001 mol %, 0.001 mol %, 0.01 mol % or 0.1 mol % or that the compound is absent.

Having now generally described the invention, the same will be more readily understood through reference to the following examples, which are provided by way of illustration and are not intended to be limiting of the present invention.

### EXAMPLES

### Example 1. Treatment of Ulcerative Colitis by MB-102 Empty Liposomes

Materials and Methods

Materials

Active compound MB-102 is composed of empty PEGylated liposomes with the lipid composition: hydrogenated soy phosphatidylcholine (HSPC) 55 mol %, cholesterol 39 mol % and n-(carbonyl-methoxypolyethyleneglycol 1,2-distearoyl-sn-glycero-3-phosphoethanol-amine (MPEG-DSPE) 6 mol %, and having an and the average diameter of about 78 nm. The liposomes were suspended in a buffer comprising 10% sucrose and 10 mM histidine, pH 6.5.

MB-102 Liposome Preparation

A mixture of HSPC, cholesterol and MPEG2000-DSPE (in 55:39:6 mole ratio) was dissolved in ethanol to a concentration of 60% w/v. This solution was injected into a buffer solution and a dispersion of 10% lipid (w/v) of multilamellar liposomes (MLV) was formed. The MLV were then downsized by sequential extrusion steps, done above the phase transition temperature, through polycarbonate filters of decreasing defined pore size, starting with 400-nm and ending with 50-nm pore-size filters under increasing nitrogen pressure (up to 200 psi), using an extruder device. The liposomes solution was then dialyzed against the buffer comprising 10% (w/v) sucrose and 10 mM histidine, pH—6.5. The final concentration of liposomes was 40 mg lipids/ml. The liposome particle size was 77.5 nm (Z average). The liposome solution was stored at 2-8° C.

Acute Inducible Colitis Model

For generation of inducible colitis model C57BL female mice, 9 weeks old (Harlan, Israel) were used. The colitis was induces by Dextran Sulfate Sodium (DSS) (2.5% w/v) in drinking water for 5 days followed by 9 days of untreated water (Perse and Cerar, Journal of Biomedicine and Biotechnology 2012, Article ID 718617).

The Study Groups:

Three groups of animal were tested group 1—healthy untreated mice (n=3); group 2 (negative control)—mice with induced colitis treated with saline (n=10); and group 3—mice with induced colitis treated with MB-102.

Experimental Procedure:

The mice were treated twice a week by intravenous injections: mice of group 2 were injected with saline: 150 μl on days 0 and 7, and 100 μl on days 3 and 10; and mice of group 3 were injected with 6 mg MB-102 on days 0 and 7, and 4 mg of MB-102 on days 3 and 10. Body weight, intestinal bleeding and stool consistency were measured daily. On day 14 the animals were sacrificed and the length of colon was measure.

Results

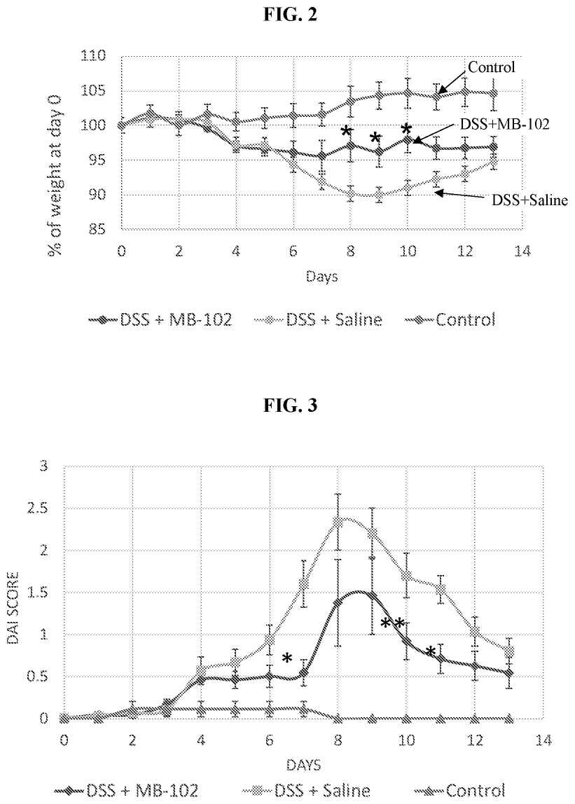

MB-102, empty PEGylated liposomes, showed a profound effect on the symptoms of colitis (in the mouse model). As can be seen from FIG. 2, MB-102 significantly reduced weight loss in the mice with induces colitis compared to the untreated mice (*p<0.05).

The Disease Activity Index (DAI) was also significantly improved for the MB-102 treated mice as compared to the untreated mice (FIG. 3). The DAI is calculated by grading on a scale of 0 to 4 the following parameters: (1) change in weight (0, <1%; 1, 1-5%; 2, 5-10%; 3, 10-15%; and 4, >15%); (2) intestinal bleeding (0, negative; 4, positive), and (3) stool consistency (0, normal; 2, loose stools; 4, diarrhea). The combined scores are then divided by 3 to obtain the final disease activity index.

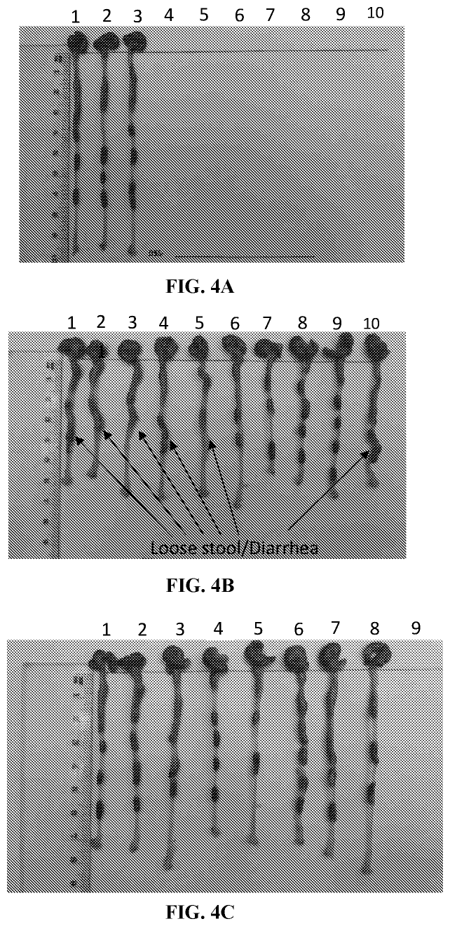

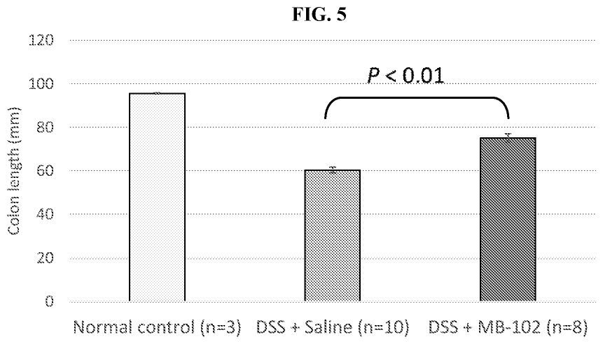

In addition, the effect of the MB-102 treatment on a length of a colon in mouse colitis model was tested. It was found that the colons of the treated mice were significantly longer than the colons of the untreated mice, although shorter than of the healthy mice (FIGS. 4 and 5 and Table 1). In addition, it can be seen from FIGS. 4B and 4C that when most of the colons of the saline treated mice (control) have the signs of loose stool, indicating for diarrhea, the content of the colons of the MB-102 treated mice was solid and resembled the content of the healthy mice and did not contain loose stool.

TABLE 1
Colon length of MB-102 treated mice with induces colitis

| | Healthy control | DSS + saline | DSS + MB-102 |
|---|---|---|---|
| | (n = 3) | (n = 10) | (n = 8) |
| Average | 95.7 | 60.3 | 75.1 |
| SEM | 0.27 | 1.33 | 1.92 |

### Example 2. Treatment of Ulcerative Colitis by MB-102, MB-103, MB-104 and MB-105 Empty Liposomes

Materials and Methods

Materials

Active compound MB-103 is composed of empty PEGylated liposomes with the lipid composition: hydrogenated soy phosphatidylcholine (HSPC) 61 mol % and cholesterol 39 mol % and having an average size of about 75 to about 85 nm. The liposomes are suspended in a buffer comprising 10% sucrose and 10 mM histidine, pH 6.5.

Active compound MB-104 is composed of empty liposomes with the lipid composition: hydrogenated soy phosphatidylcholine (HSPC) and having an average size of about 75 to about 85 nm. The liposomes are suspended in a buffer comprising 10% sucrose and 10 mM histidine, pH 6.5.

Active compound MB-105 is composed of empty PEGylated liposomes with the lipid composition: hydrogenated soy phosphatidylcholine (HSPC) 94 mol % and MPEG2000-DSPE 6 mol %, and having an average size of about 75 to about 85 nm. The liposomes are suspended in a buffer comprising 10% sucrose and 10 mM histidine, pH 6.5.

MB-103, MB-104 and MB-105 Liposome Preparation

Active compounds MB-102 is prepared as described in Example 1 but at a concentration of 60 mg/ml. A mixture of HSPC and cholesterol (in 61:39 mole ratio)(MB-103), HSPC only (MB-104) or a mixture of HSPC and MPEG2000-DSPE (in 94:6 mole ratio) (MB-105) is dissolved in ethanol to a concentration of 60% w/v. This solution is injected into a buffer solution and a dispersion of 10% lipid (w/v) of multilamellar liposomes (MLV) is formed. The MLV is then downsized by sequential extrusion steps, done above the phase transition temperature, through polycarbonate filters of decreasing defined pore size, starting with 400-nm and ending with 50-nm pore-size filters under increasing nitrogen pressure (up to 200 psi), using an extruder device. The liposomes solution is then dialyzed against the buffer comprising 10% (w/v) sucrose and 10 mM histidine, pH—6.5. The final concentration of liposomes is 60 mg lipids/ml. The liposome particle size is about 80 nm. The liposome solutions are stored at 2-8° C.

Mice with acute inducible colitis are generated as in Example 1.

The Study Groups:

Sixteen groups of animal are tested. Group 1—healthy untreated mice (n=3); group 2-4 (negative controls)—mice with induced colitis treated with saline (n=10 each); group 5-7 (n=10 each) mice with induced colitis and being treated with MB-102, groups 8-10 (n=10 each) mice with induced colitis and being treated with MB-103, groups 11-13 (n=10 each) mice with induced colitis and being treated with MB-104 and groups 14-16 (n=10 each) mice with induced colitis and being treated with MB-105.

Experimental Procedure:

The mice are treated twice a week, on days 0, 3, 7 and 10 by intravenous (IV) injections, subcutaneous (SC) injections or intrarectal administrations. Mice of group 2-4 are injected/administrated with 10% (w/v) sucrose and 10 mM histidine, pH—6.5 (100 μl). Mice of group 5-7 are injected/administrated with 6 mg of MB-102. Mice of group 8-10 are injected/administrated with 6 mg of MB-103. Mice of group 11-13 are injected/administrated with 6 mg of MB-104 and mice of group 14-16 are injected/administrated with 6 mg of MB-105. Groups 2, 5, 8, 11 and 14 are injected IV. Groups 3, 6, 9, 12 and 15 are injected SC. Groups 4, 7, 10, 13 and 16 are treated by intrarectal administrated. Body weight, intestinal bleeding and stool consistency are measured daily. On day 14, the animals are sacrificed and the length of colon and its content is examined.

### Example 3. Treatment of Crohn's Disease by MB-102, MB-103, MB-104 and MB-105 Empty Liposomes

Materials and Methods

Active compounds MB-102, MB-103, MB-104 and MB-105 are prepared as described in Example 2.

Inducible Crohn's Disease Model

For generation of inducible model of Crohn's disease—C57BL female mice, 9 weeks old are administered with 200 mg/kg of trinitrobenzene sulfonic acid (TNBS) dissolved in 30% ethanol. The TNMS is instilled in mice using a medical-grade polyurethane catheter (external diameter 2 mm) approximately 3-4 cm proximal to the anus.

The Study Groups

Sixteen groups of animal are tested. The tested groups of animal are as following: Group 1—healthy untreated mice (n=3); group 2-4 (negative controls)—mice with induced Crohn's disease treated with 10% (w/v) sucrose and 10 mM histidine, pH—6.5 (n=10 each); group 5-7 (n=10 each) mice with induced Crohn's disease and being treated with MB-102, groups 8-10 (n=10 each) mice with induced Crohn's disease and being treated with MB-103, groups 11-13 (n=10 each) mice with induced Crohn's disease and being treated with MB-104 and groups 14-16 (n=10 each) mice with induced Crohn's disease and being treated with MB-105.

Experimental Procedure:

The mice are treated twice a week, on days 0, 3, 7 and 10 by intravenous (IV) injections, subcutaneous (SC) injections or intrarectal administrations. Mice of group 2-4 are injected/administrated with 10% (w/v) sucrose and 10 mM histidine, pH—6.5 (100 μl). Mice of group 5-7 are injected/administrated with 6 mg of MB-102. Mice of group 8-10 are injected/administrated with 6 mg of MB-103. Mice of group 11-13 are injected/administrated with 6 mg of MB-104 and mice of group 14-16 are injected/administrated with 6 mg of MB-105. Groups 2, 5, 8, 11 and 14 are injected IV. Groups 3, 6, 9, 12 and 15 are injected SC. Groups 4, 7, 10, 13 and 16 are treated by intrarectal administrated. Body weight, intestinal bleeding and stool consistency are measured daily. On day 14, the animals are sacrificed and the length of colon and its content is examined.

### Example 4. Empty Liposomes MB-102, MB-103, MB-104 and MB-105 for Treating of Multiple Sclerosis (MS)

Material and Methods

Active compounds MB-102, MB-103, MB-104 and MB-105 are prepared as described in Examples 1 and 2.

MS Model

Experimental allergic encephalomyelitis (EAE) in the most used animal model of multiple sclerosis model. Dark agouti (DA) (Harlan, Italy) female rats, 150-180 g. body weight, are used for the purpose of generation of the model. The rats are sensitized by a medium containing 0.15 mg/ml guinea pig spinal cord tissue in complete Freund's adjuvant (CFA, Sigma, Saint Louse, USA), 50% v/v, to which 5 mg/ml of heat-inactivated Mycobacterium tuberculosis (Difco H37Ra, DB, Milan, Italy) is added. Sensitization is performed by injecting 100 μl in both hind pads. Control rats (n=10) and adjuvant-injected rats (CFA, 50% v/v, heat-inactivated M. tuberculosis, 5 mg/ml) (n=10 in each group) are used. The rats are weighed daily and examined for clinical signs of EAE, according to the following semi quantitative score for neurological disability: 0=no signs, 1=loss of tail tone=2, mono or bilateral weakness of hind legs or middle ataxia, 3=ataxia or paralysis, 4=severe hind legs paralysis, and 5=severe hind leg paralysis and urinary incontinence. In view of the animals' disability, wet food was included inside the cages to facilitate feeding. The study groups:

The tested groups of animal are as following: Group 1—healthy untreated rats (n=5); group 2 (negative control)—rats with induced EAE treated IV with 10% (w/v) sucrose and 10 mM histidine, pH—6.5 (n=10); group 3-5 (n=10 each) rats with induced EAE and being treated with MB-102 IV (group 3) SC (group 4) or orally (group 5); groups 6-8 (n=10 each): rats with induced EAE and being treated with MB-103 IV (group 6), SC (group 7) or orally (group 8); groups 9-11 (n=10 each): rats with induced EAE and being treated with MB-104 IV (group 9), SC (group 10) or orally (group 11), and groups 12-14 (n=10 each): rats with induced EAE and being treated with MB-105 IV (group 12), SC (group 13) or orally (group 14)

Experimental Procedure:

The rats are treated twice a week via intravenous, subcutaneous or oral administration: rats of group 2 are injected IV with 10% (w/v) sucrose and 10 mM histidine, pH—6.5: 100 μl on days 0, 3, 7, 10, 14, 17 and 21; group 3-5, 6-8, 9-11 and 12-14 are administrated with 6 mg MB-102, MB-103, MB-104 or MB-105, respectively, on days 0, 3, 7, 10, 14, 17 and 21 as follows: Rats of groups 3, 6, 9 and 12 are IV injected. Rats of groups 4, 7, 10 and 13 are SC injected, and rats of groups 5, 8, 11 and 14 are orally administrated. Rats are scored for clinical signs of EAE and weighed daily until day 40.

Although the present invention has been described herein above by way of preferred embodiments thereof, it can be modified, without departing from the spirit and nature of the subject invention as defined in the appended claims.

1.-41. (canceled)

42. A method of treatment of a gastrointestinal autoimmune disease in a subject in need thereof comprising administering a pharmaceutically acceptable composition comprising a therapeutically effective amount of small unilamellar vesicle (SUV) liposomes, wherein said composition is substantially devoid of a compound being an active agent in a medication prescribed for treatment of a gastrointestinal autoimmune disease.

43. The method of claim 42, wherein the gastrointestinal autoimmune disease is inflammatory bowel disease (IBD) or irritable bowel syndrome (IBS).

44. The method of claim 43, wherein the IBD is selected from ulcerative colitis and Crohn's disease.

45. The method of claim 42, wherein said liposomes comprise a liposome forming lipid selected from the group consisting of a phosphatidylcholine, phosphatidylinositol, phosphatidylglycerol, phosphatidylethanolamine, I-palmitoyl-2-oleoylphosphatidyl choline (POPC), sphingophospholipids, distearoyl, and any combination thereof.

46. The method of claim 45, wherein said phosphatidylcholine is hydrogenated soy phosphatidylcholine (HSPC); and/or said phosphatidylethanolamine is 1,2-distearoyl-sn-glycero-3-phosphoethanolamine (DSPE); and/or said distearoyl is distearoyl glycol (DSG) or oxycarbonyl-3-amino-1,2-propanediol distearoylester (DS).

47. The method according to claim 42, wherein said liposomes comprise stabilizing polymer molecules.

48. The method of claim 47, wherein said stabilizing polymer molecules are selected from the group consisting of polyalkylether, polysialic acid, polylactic acid and polyglycolic acid.

49. The method of claim 48, wherein said polyalkylether is polyethylene glycol (PEG).

50. The method of claim 49, wherein the polyethylene glycol molecules has an average molecular weight of about 350 Da to about 100,000 Da, about 1000 Da to about 10000 Da, about 1500 Da to about 5000 Da, or about 2000 Da.

51. The method of claim 49, wherein said liposomes comprise about 0.01 mol % to about 20 mol % of PEG.

52. The method of claim 42, wherein the liposomes are substantially devoid of cholesterol.

53. The method of claim 42, wherein the liposomes further comprising cholesterol.

54. The method of claim 53, wherein said liposomes comprise about 0% to about 50% cholesterol.

55. The method of claim 42, wherein said liposomes comprise (i) HSPC and cholesterol, (ii) HSPC, cholesterol and MPEG-DSPE (iii) phosphatidylcholine and DSPE or (iv) HSPC and MPEG-DSPE.

56. The method of claim 55, wherein said liposomes comprise (i) about 40 mol % to about 80 mol % HSPC and about 20 mol % to about 60 mol % cholesterol or (ii) about 40 mol % to about 60 mol % HSPC, about 30 mol % to about 50 mol % cholesterol and about 2 mol % to about 10 mol % MPEG-DSPE, optionally wherein the methyl polyethyleneglycol (MPEG) of DSPE has a molecular weight of about 1000 Da to about 5000 Da, or about 2000 Da.

57. The method of claim 42, wherein the average diameter of said liposomes is about 10 to about 300 nm, about 50 to about 100 nm, about 60 nm to about 90 nm, or about 70 nm to 85 nm.

58. The method of claim 42, wherein said liposomes are essentially devoid of a compound selected from the group consisting of an anti-inflammatory compound, non-steroidal anti-inflammatory compound, an active agent in a prescription drug, a compound prescribed for treatment of an autoimmune disease, and a compound prescribed for treatment of IBD, IBS, Crohn's disease or ulcerative colitis.

59. The method of claim 42, wherein said liposomes are drug-free liposomes.

60. The method of claim 42, wherein said liposomes further comprise one or more non-active compound such as vitamins, minerals, salt, food additive, antioxidant, pharmaceutically acceptable excipients and nutraceuticals.

61. The method of claim 42, wherein the pharmaceutically acceptable composition is orally, rectally or parenterally administered, optionally wherein the pharmaceutically acceptable composition comprising about 10 to about 200, or about 50 to about 120 mg/ml of liposomes.
